(19) United States
(12) Patent Application Publication
(10) Pub. No.: US 2025/0206645 A1
(43) Pub. Date: Jun. 26, 2025

(54) METHOD AND APPARATUS FOR PRODUCING UREA-FREE WATER, AND METHOD AND APPARATUS FOR ANALYZING UREA

(71) Applicant: ORGANO CORPORATION, Tokyo (JP)

(72) Inventors: Yui SHIOYA, Tokyo (JP); Kazushige TAKAHASHI, Tokyo (JP)

(73) Assignee: ORGANO CORPORATION, Tokyo (JP)

(21) Appl. No.: 18/845,505

(22) PCT Filed: Feb. 10, 2023
(86) PCT No.: PCT/JP2023/004516

§ 371 Date: Sep. 10, 2024

(30) Foreign Application Priority Data
Mar. 11, 2022 (JP) 2022-038018

(51) Int. Cl. C02F 3/34; B01D 61/02; C02F 1/44; C02F 101/38; G01N 33/18

(52) U.S. Cl. CPC C02F 3/342; B01D 61/025; C02F 1/441; G01N 33/1826; C02F 2101/38; C12Y 305/01005

(57) ABSTRACT

Provided are a method and an apparatus for producing urea-free water that can be used when analyzing urea in sample water, and that can efficiently produce urea-free water in which the urea concentration is reduced to at least a level that does not affect the analysis. The method for producing urea-free water includes adding urease to urea-containing water, thereby decomposing urea, to obtain treated water containing urease, and passing treated water through separation membrane, thereby removing the urease, to obtain urea-free water.

### TECHNICAL FIELD

The present invention relates to a quantification of urea in water. In particular, it relates to a method and apparatus for producing urea-free water used when quantifying urea, and a method and apparatus for quantifying urea in which the urea-free water is used.

### BACKGROUND ART

There is a demand for accurate analysis and quantification of trace amounts of urea in water. For example, when producing pure water from raw water by a pure water production system, it is difficult to remove urea from the raw water using an ion exchange apparatus and an ultraviolet oxidation apparatus that constitute the pure water production system. Therefore, it is necessary to remove urea from raw water before supplying it the pure water production system. As a method for removing urea, a method is known in which a chemical agent that generates hypobromous acid is added to raw water and urea is selectively oxidized by the hypobromous acid. However, since the above chemical agent impose a burden on the pure water production system, it is preferable to use a small amount of the chemical agent. Therefore, it is desirable to determine the concentration of urea in raw water in advance, and then add an appropriate amount of the chemical agent. Furthermore, there is a need to measure the concentration of urea in pure water produced from the pure water production system.

Known methods for quantifying urea include a method based on a colorimetry which uses diacetyl monoxime (for example, the method described in the document regarding Standard methods of analysis for hygienic chemists (Non-Patent Literature 1)). In the colorimetry using diacetyl monoxime, other reagents (for example, antipyrine sulfuric acid solution, aqueous solution of semicarbazide hydrochloride, aqueous solution of manganese chloride and potassium nitrate, sodium dihydrogen phosphate sulfuric acid solution, etc.) can be used in combination for the purpose of promoting the reaction. When antipyrine is used in combination, diacetyl monoxime is first dissolved in an acetic acid solution to prepare a diacetyl monoxime acetic acid solution. Next, antipyrine (1,5-dimethyl-2-phenyl-3-pirazolone) is dissolved in, for example, sulfuric acid to prepare an antipyrine-containing reagent solution. The diacetyl monoxime acetic acid solution and the antipyrine-containing reagent are sequentially mixed with a sample water, and the absorbance at a wavelength of about 460 nm is measured, and quantification is performed by comparison with a standard solution.

However, the method for quantifying urea using colorimetry with diacetyl monoxime is intended for the purpose of quantifying urea in, for example, pool water or public bath water. Hence, the sensitivity is poor for quantifying urea in raw water for supplying to a pure water production process. Patent Literature 1 discloses a method for continuously and online quantifying urea in a sample water in a concentration range from below ppb to several ppm by measuring absorbance by applying a flow injection analysis based on colorimetry using diacetyl monoxime. Patent literature 2 discloses that when quantifying urea by applying a flow injection analysis based on a colorimetry method using diacetyl monooxime, continuous automatic measurement can be stably carried out online over a long period of time by refrigerating a reagent used for the reaction.

### CITATION LIST

### Patent Literature

Patent Literature 1: JP2000-338099A
Patent Literature 2: JP2018-179545A
Patent Literature 3: JPH6-86997A

### Non-Patent Literature

Non-Patent Literature 1: The Pharmaceutical Society of Japan (ed.), Methods of Analysis in Health Science—with commentary—1990, 4. 1. 2. 3 (13) 1 (1990 edition, 4th printing with supplement (1995), p. 1028) 1995

### SUMMARY OF INVENTION

### Technical Problem

The methods described in Patent Literatures 1 and 2 are techniques are capable of quantifying urea with high sensitivity by using flow injection analysis. However, if urea is contained in the water used in these quantification methods, such as a water used for preparing a concentration standard solution and a carrier water used in flow injection analysis, the urea contained in the water can cause analytical errors. Even when urea is quantified by a method other than flow injection analysis, the presence of urea in any of the waters used in operations of the quantification will be a factor of analytical error. For example, even when performing analysis by liquid chromatography, if urea is contained in the carrier water used as a mobile phase, an analytical error occurs. In particular, when analyzing trace amounts of urea in a sample water at μg/L level, if urea is present during preparation of a standard solution or if urea is present in carrier water even at μg/L level, the analysis is affected. Therefore, there is a need for water that contains almost no urea (called urea-free water).

As a method for producing urea-free water that can be used for analyzing urea in a sample water, there is a method for producing urea-free water in which water is passed through a column on which urease is immobilized to decompose urea. Additionally, as for decomposition of urea using immobilized urease, JPH6-86997A discloses a pure water production apparatus in which urea is decomposed by using a urease-supported decomposition apparatus in which urease is supported on activated carbon. However, the process for preparing immobilized urease is complicated, and it is difficult to increase the capacity of carrier for the immobilization. Consequently, it is difficult to produce a large amount of urea-free water at one time. In addition, when preparing the immobilized enzyme, a highly concentrated urease solution is brought into contact with carrier, but not all urease binds the carrier, and accordingly some of the expensive urease is wasted. Furthermore, since during passing liquid through the immobilized urease, the urease gradually detaches from the carrier, the urea decomposition performance deteriorates over time.

An object of the present invention is to provide a method and apparatus for producing urea-free water that can be used when analyzing urea in a sample water and that can efficiently produce urea-free water in which the urea concentration is reduced to a level that does not affect the analysis at least. Another objective of the present invention is to provide an analysis apparatus that can accurately quantify trace amounts of urea in a sample water by using the urea-free water.

### Solution to Problem

According to an aspect of the present invention, there is provided a method for producing urea-free water, comprising:
adding urease to a urea-containing water, thereby decomposing urea, to obtain a treated water containing urease, and
passing the treated water through a separation membrane, thereby removing the urease, to obtain a urea-free water.

According to another aspect of the invention, there is provided an apparatus for producing urea-free water, comprising:
a treatment means for decomposing urea by adding urease to a urea-containing water,
a separation membrane for removing the urease by passing a treated water obtained by the treatment means through the separation membrane,
wherein the water passing through the separation membrane is made into a urea-free water.

According to another aspect of the invention, there is provided a method for quantifying urea in sample water, comprising using a urea-free water produced by the above method for producing urea-free water, as at least one of a water to be used in preparation of a urea standard solution, and a carrier water.

According to another aspect of the invention, there is provided an apparatus for analyzing urea, by introducing a constant amount of sample water into a flow of carrier water to quantify urea in the sample water,
the apparatus for analyzing urea comprising the above apparatus for producing urea-free water,
wherein a urea-free water having a urea concentration of less than 1 μg/L obtained from the apparatus for producing urea-free water is used as the carrier water.

### Advantageous Effects of Invention

According to the present invention, it is possible to provide a method and apparatus for producing urea-free water that can be used when analyzing urea in a sample water and that can produce urea-free water in which the urea concentration is reduced to a level that does not affect the analysis at least by directly adding urease to urea-containing water. Also, it is possible to provide an analysis apparatus that can accurately quantify trace amounts of urea in a sample water by using the urea-free water.

### BRIEF DESCRIPTION OF DRAWINGS

FIG. 1 is a diagram illustrating an example of an apparatus for producing urea-free water according to the present invention.

FIG. 2 is a diagram illustrating another example of the apparatus for producing urea-free water according to the present invention.

FIG. 3 is a diagram illustrating an example of the apparatus for producing urea-free water according to the present invention, in which a reverse osmosis membrane is can be cleaned.

FIG. 4 is a diagram illustrating an example of an apparatus for analyzing urea according to the present invention, in which the urea-free water is used.

### DESCRIPTION OF EMBODIMENTS

A method for producing urea-free water according to an exemplary embodiment of the present invention is to produce urea-free water in which urea concentration is reduced to a level that does not affect an analysis of urea in sample water by treating water that may contain urea with urease. The urea-free water in which urea concentration is reduced to a level that does not affect an analysis of urea in sample water refers to a water in which the urea concentration is less than 1 μg/L, particularly less than 0.5 μg/L.

According to an exemplary embodiment of the present invention, by adding a solution in which urease is dissolved or urease itself to urea-containing water, thereby decomposing urea, to obtain a urea-free treated water. Furthermore, by removing urease from the treated water, it is possible to obtain a urea-free water that can be used as a standard solution or carrier water for quantifying trace amounts of urea in a sample water. If urease is not sufficiently removed from the treated water, urease will be mixed in with the urea-free water. When urease is mixed in with the urea-free water, the urease will affect the quantitative value of urea.

Specifically, when urea-free water containing urease is used to prepare a urea standard solution, the urease in the urea-free water decomposes and removes the urea in the urea standard solution. As a result, linearity of the calibration line cannot be obtained, making it impossible to quantitatively determine the amount of urea. Also, if urea-free water containing urease is used as carrier water into which a sample water is injected, the urease in the urea-free water will decompose and remove the urea in the sample water. The amount as the quantification result will be smaller than the original amount of urea in the sample water. Whether or not urease is mixed in with the carrier water can be checked by adding urea to the carrier water at, for example, 50 ppb or 100 ppb, leaving it at room temperature for several days, and then checking for the presence or absence of urea decomposition due to leakage of urease. Therefore, when urea-free water is produced by adding a solution in which urease is dissolved or urease itself to urea-containing water, it is necessary to ensure that urease is not mixed in with the urea-free water.

The method for producing urea-free water according to the present invention includes a treatment step of adding urease to urea-containing water, thereby decomposing urea, to obtain a treated water containing urease, and a separation step of passing the treated water through a separation membrane, thereby removing the urease, to obtain a urea-free water.

Hereinafter, exemplary embodiments according to the present invention will be described with reference to the drawings. However, the present invention is not limited to these exemplary embodiments.

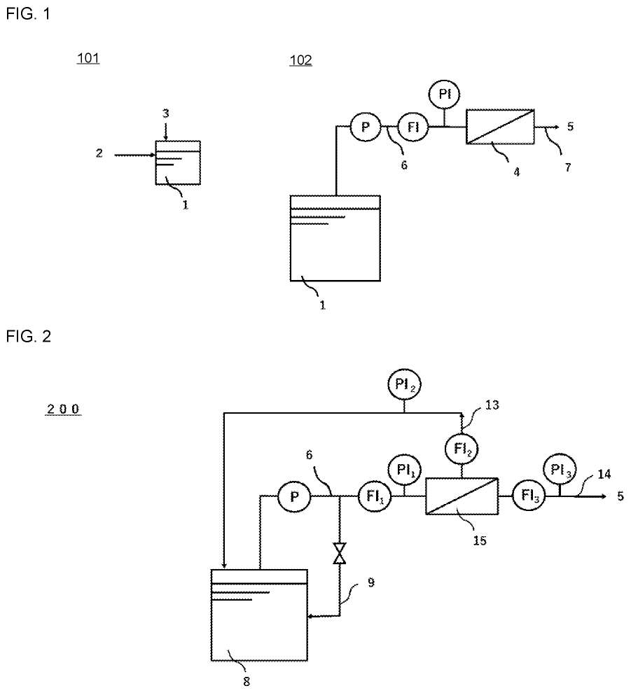

FIG. 1 shows an example of an apparatus for producing urea-free water, including treatment means 101 for obtaining treated water 1 by adding urease 3 to urea-containing water 2, and separation means 102 for obtaining urea-free water 5 that does not contain urease by passing treated water 1 through separation membrane 4.

In treatment means 101, urease 3 is added to urea-containing water 2 to obtain treated water 1, in which urea in urea-containing water 2 is decomposed.

The decomposition of urea by urease is a hydrolysis reaction with urease as an enzyme (catalyst) and is represented by the following formula. Urease can decompose even minute amounts of urea into carbon dioxide and ammonia in the presence of water.

(NH₂)₂CO+H₂O→CO₂+NH₃

Urease 3 to be added may be in a solid state or in a solution state dissolved in a solvent. The solvent for dissolving urease 3 is not particularly limited as long as it does not affect urease and does not affect the quantification analysis of urea using urea-free water obtained through separation means 102, but water is preferable. The urease may be added directly to a tank containing the urea-containing water, or a line for adding urease may be provided on the line through which the urea-containing water flows. In addition, after addition of urease, sufficient time is required for urea to be decomposed, and the time can be appropriately set depending on the expected urea concentration in the urea-containing water, the amount of urease added, the water temperature, the degree of stirring, etc. Also, urease can be added preliminarily to determine the time required for the urea concentration to fall below the detection limit, and actual production of treated water can be carried out by leaving it stationary or with stirring for that time or longer. As the stirring method, a conventional method can be used. For example, mixing by inversion or manual stirring may be used. Alternatively, the treated water may be circulated and agitated until the urea is sufficiently decomposed. In the present invention, the urea decomposition treatment step may be carried out in a batch system in which treatment means 101 and separation means 102 are separate, or in treatment means 101 combined separation means 102. It is preferable to conduct urea decomposition treatment with urease in the batch system in terms of ensuring sufficient treatment time and in terms of the treatment amount. For example, urease can be added to a tank containing urea-containing water and left to treat overnight to fully decompose the urea.

In separation means 102, the treated water 1 obtained by treatment means 101 is passed through supply line 6 to separation membrane 4, where urease is separated by separation membrane 4, and then urea-free water 5 not containing urease is obtained via drainage line 7. Supply line 6 is equipped with pump P for passing treated water 1 through separation membrane 4, flow meter FI, and pressure gauge PI. If the pressure when passing through separation membrane 4 exceeds the withstand pressure of the vessel in which the separation membrane is loaded, a return line may be provided before separation membrane 4 to return treated water 1 before passing it through separation membrane 4. Separation membrane 4 is not particularly limited as long as it does not allow urease to permeate, but is preferably a reverse osmosis membrane. According to the studies of the present inventors, it has been confirmed that even in a nanofiltration membrane (NF membrane) having a molecular weight cut-off sufficiently smaller than the molecular weight of urease, a small amount of urease leaks out. When a reverse osmosis membrane is used as the separation membrane, the reverse osmosis membrane preferably has the salt rejection rate of 90% or more. There is no particular upper limit, and 100% may be acceptable if possible, but the upper limit is practically 99.7%, and 99.0% is sufficient from the standpoint of ease of production. Here, the salt rejection rate of 90% or more means that the salt rejection rate of a NaCl aqueous solution is 90% or more when the NaCl aqueous solution having the concentration of 250 ppm is treated at 25° C., pH7, the recovery rate of 15%, and the inlet pressure of 0.34 MPa. The salt rejection rate was calculated by measuring the Na concentration and the Cl concentration of the NaCl aqueous solution before and after permeation through the separation membrane by ion chromatography and dividing the difference in concentration before and after permeation by the concentration before permeation. Alternatively, it can be calculated by determining the conductivities of the feed water and the permeate water, and dividing the difference between the two conductivities by the conductivity of the feed water. When a reverse osmosis membrane in the above range is used, the water can be separated into permeate water that does not contain urease and concentrated water that does contain urease. In addition, the concentrated water can be circulated to the treated water 1 and passed through the reverse osmosis membrane again. In order to prevent contamination with bacteria, an ultraviolet (UV) irradiation device may be provided in the line through which the permeate water passes. Furthermore, in order to further remove urease, a means for passing the permeate water through an anion exchange resin by any method may be provided downstream of separation membrane 4. The method of passing permeate water through an anion exchange resin includes passing the water through a column packed with the anion exchange resin. As the anion exchange resin, either a strong basic anion exchange resin or a weak basic anion exchange resin can be used. When used in combination with a UV irradiation device, a means for passing the water through the anion exchange resin can be provided upstream or downstream of the UV irradiation device. From the viewpoint of preventing bacteria from mixing with urea-free water 5 that does not contain urease, it is preferable to provide the means for passing the water through the anion exchange resin upstream of the UV irradiation device. The reverse osmosis membrane may be of a multi-stage type.

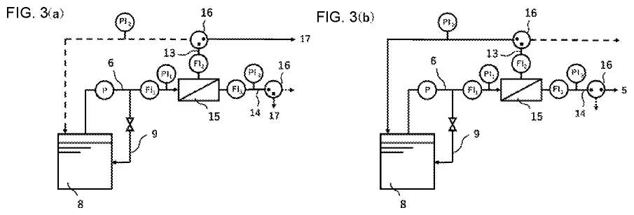

FIG. 2 shows an example of the apparatus for producing urea-free water capable of separating treated water 8 containing urease, in which urea is decomposed, into permeate water containing no urease and concentrated water containing urease by passing the treated water through reverse osmosis membrane 15.

The apparatus for producing urea-free water 200 shown in FIG. 2 has reverse osmosis membrane 15. This reverse osmosis membrane 15 preferably has a salt rejection rate of 90% or more, similar to the reverse osmosis membrane used as separation membrane 4 in shown FIG. 1. From the above reverse osmosis membrane 15, two lines are provided: concentrated water line 13 for taking out concentrated water containing urease, and permeate water line 14 for discharging permeate water containing no urease. Treated water 8, which contains urease and in which urea is decomposed, is passed through supply line 6 to reverse osmosis membrane 15. As treated water 8, treated water 1 obtained by treatment means 101 shown in FIG. 1 can also be used. Supply line 6 is equipped with pump P for passing treated water 8 through reverse osmosis membrane 15, flow meter FI₁, and pressure gauge PI₁. If the pressure when passing through reverse osmosis membrane 15 exceeds the withstand pressure of the vessel in which reverse osmosis membrane 15 is loaded, return line 9 may be provided before reverse osmosis membrane 15 to return treated water 8 before passing it through reverse osmosis membrane 15. Concentrated water line 13 is equipped with flow meter FI₂ and pressure gauge PI₂, and the concentrated water is passed through concentrated water line 13, returned to the tank for treated water 8, and is passed through reverse osmosis membrane 15 again. In addition, permeate water line 14 is equipped with flow meter FI₃ and pressure meter PI₃, and urea-free water 5 not containing urease is obtained. In order to prevent contamination with bacteria, a UV irradiation device may be provided in permeate water line 14. Furthermore, in order to further remove urease, a means for passing the permeate water through an anion exchange resin by any method may be provided downstream of reverse osmosis membrane 15. The method of passing permeate water through an anion exchange resin includes passing the water through a column packed with the anion exchange resin. As the anion exchange resin, either a strong basic anion exchange resin or a weak basic anion exchange resin can be used. When used in combination with a UV irradiation device, a means for passing the water through the anion exchange resin can be provided upstream or downstream of the UV irradiation device. From the viewpoint of preventing bacteria from mixing with urea-free water 5 that does not contain urease, it is preferable to provide the means for passing the water through the anion exchange resin upstream of the UV irradiation device. The reverse osmosis membrane may be of a multi-stage type.

FIG. 3 shows an example of the apparatus for producing urea-free water capable of cleaning reverse osmosis membrane 15 by discharging permeate water and concentrated water as waste liquid 17 for a certain period of time when using an unused reverse osmosis membrane.

When unused reverse osmosis membrane 15 is used in the present invention, it is desirable to clean it before use. Using without cleaning may affect the measurement of urea concentration. Therefore, before starting the separation of urease using reverse osmosis membrane 15, it is necessary to clean reverse osmosis membrane 15. Specifically, three-way valve 16 is provided in each of concentrated water line 13 and permeate water line 14 in shown FIG. 2 so that waste liquid 17 can be discharged from each line. First, as shown in FIG. 3(a), treated water 8 containing urease is passed through reverse osmosis membrane 15, and the permeate water and the concentrated water are discharged as waste liquid 17 for a certain period of time to clean reverse osmosis membrane 15 so that the measurement of urea concentration is not affected. Next, three-way valve 16 is switched as shown in FIG. 3(b), so that the concentrated water containing urease is circulated, while urea-free water 5 containing no urease, which is the permeate water, is collected. As treated water 8 containing urease, treated water 1 obtained by treatment means 101 shown in FIG. 1 can also be used. The cleaning liquid for cleaning reverse osmosis membrane 15 is not particularly limited as long as it does not contain urea and does not contain any substance that interferes with the analysis of urea. The method for cleaning reverse osmosis membrane 15 is not particularly limited as long as the measurement of urea concentration is not affected when reverse osmosis membrane 15 is in use.

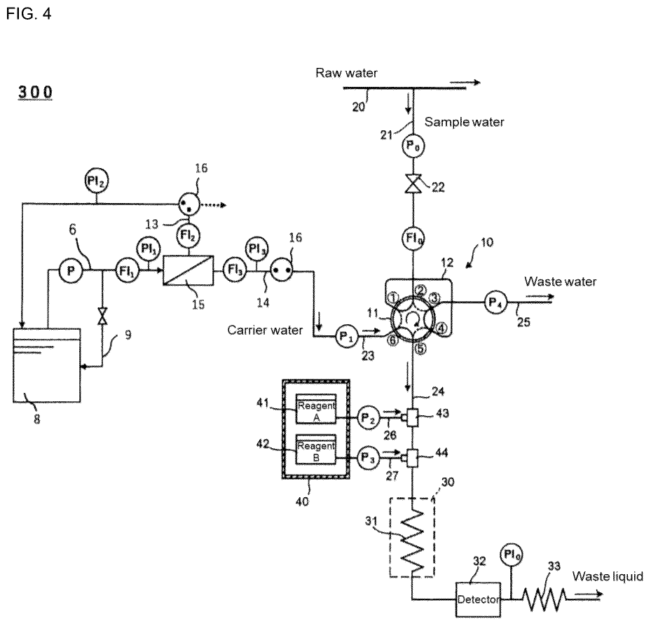

FIG. 4 shows apparatus for analyzing urea 300 to which the method for producing urea-free water according to the present invention is applied. Here, an example will be described in which raw water used in pure water production or pure water itself is used as sample water, and trace amounts of urea contained in the sample water are continuously quantified online. The sample water to be analyzed for urea is not limited to raw water used in pure water production or pure water.

As shown in FIG. 4, line 20 of raw water used for pure water production is provided, and the raw water is pumped through line 20 by pump P₀. Sample water line 21 is provided branching off from raw water line 20. Sample water line 21 is a line of sample water branched off from the raw water, and is equipped with opening/closing valve 22 and flow meter FI₀. Sampling valve 10 (also called an injector or injection valve) is provided at the end of sample water pipe 21. The portion downstream from sampling valve 10, including sampling valve 10, is configured as a flow injection analysis apparatus and is actually involved in the quantification of urea.

Sampling valve 10 is of a configuration commonly used in flow injection methods, and includes six-way valve 11 and sample loop 12. Six-way valve 11 has six ports indicated by circled numbers in the figure. Sample line 21 is connected to the port (2). Line 23 through which carrier water is supplied via pump P₁ is connected to the port (6), and line 25 through which sample water is discharged via pump P₄ is connected to the port (3). Sample loop 12 for collecting a predetermined volume of sample water is connected between the port (1) and the port (4). One end of line 24 serving as an outlet of sampling valve 10 is connected to the port (5). Carrier water is supplied to pump P₁ via line 19 and is pumped from pump P₁ via line 23 toward the port (6).

As the carrier water, pure water or the like is generally used, but it has a significant effect on the accuracy of urea quantification, and hence it is required that the urea concentration be extremely low. When quantifying urea at the μg/L level in sample water, the urea concentration in the carrier water should be less than 1 μg/L. Therefore, in the exemplary embodiment, urea-free water produced by the production method according to the present invention is used as the carrier water. To this end, the analyzing apparatus includes reverse osmosis membrane 15. Treated water 8 containing urease in which urea is decomposed is passed through supply line 6 to reverse osmosis membrane 15. Supply line 6 is provided with pump P for sending treated water 8 to reverse osmosis membrane 15, flow meter FI₁, and pressure gauge PI₁. As treated water 8, separately from analyzing apparatus 300, a treated water is used which is obtained by adding urease to urea-containing water in advance to decompose the urea in the urea-containing water. In order to prevent the pressure when pumping to reverse osmosis membrane 15 from exceeding the withstand pressure of the vessel in which reverse osmosis membrane 15 is loaded, return line 9 is provided in front of reverse osmosis membrane 15 so that treated water 8 containing urease before passing through reverse osmosis membrane 15 can be circulated. Furthermore, two lines are provided from reverse osmosis membrane 15: concentrated water line 13 through which concentrated water containing urease passes, and permeate water line 14 through which permeate water not containing urease passes. Concentrated water line 13 and permeate water line 14 are each provided with three-way valve 16, so that the concentrated water and the permeate water can each be discharged as waste liquid. As a result, when unused reverse osmosis membrane 15 is used, reverse osmosis membrane 15 can be cleaned by first discharging for a certain period of time to ensure that the measurement of the urea concentration is not affected. Concentrated water line 13 is provided with flow meter FI₂ and pressure gauge PI₂. The concentrated water is circulated through concentrated water line 13 to treated water 8 containing urease before being passed through reverse osmosis membrane 15, and is passed through reverse osmosis membrane 15 again. Furthermore, permeate water line 14 is provided with flow meter FI₃ and pressure meter PI₃, and urease-free water is supplied to line 23 as carrier water and used in the quantification of urea by flow injection analysis.

If the connection between port X and port Y in six-way valve 11 is represented as (X-Y), six-way valve 11 can switch between the first state of (1-2), (3-4), (5-6) and the second state of (2-3), (4-5), (6-1). In FIG. 4, the connections between the ports in the first state are shown by solid lines, and the connections between the ports in the second state are shown by dashed lines. In the first state, the carrier water flows from line 23->port (6)->port (5)->line 24 and flows out downstream from sampling valve 10. The sample water flows from sample water line 21->port (2)->port (1)->sample loop 12->port (4)->port (3) and is discharged from line 25 as waste water. When switching from the first state to the second state, the sample water flows from sample water line 21->port (2)->port (3) and is discharged from line 25, and the carrier water flows from line 23->port (6)->port (1)->sample loop 12->port (4)->port (5)->line 24 and flows out downstream. At this time, the sample water that has already flowed in and filled sample loop 12 when in the first state flows into line 24 from port (5) prior to the carrier water, and flows downstream of sampling valve 10. The volume of the sample water flowing in line 24 is determined by sample loop 12. Thus, by repeatedly switching between the first state and the second state, for example by rotating six-way valve 11 in the direction of the illustrated arrow, a predetermined volume of the sample water can be repeatedly sent into line 24. The first state and the second state can be switched at predetermined time intervals, taking into consideration the residence time required for the reaction and the time it takes for the urea to be detected by detector 32. Also, switching can be performed by detecting that the sample water introduced into detector 32 has been discharged from detector 32. In this way, by automatically switching between the first state and the second state, urea can be continuously quantified.

In the analyzing apparatus, the flow injection method is applied to the quantification of urea by a colorimetric method using diacetyl monoxime. Therefore, as reaction reagents for the quantification of urea, a diacetylmonoxime acetate solution (hereinafter referred to as reagent A) and an antipyrine-containing reagent solution (hereinafter also referred to as reagent B) are used. Here, a case will be described in which the antipyrine-containing reagent solution is used as a reagent to be used in combination with diacetyl monoxime, but the reagent to be used in combination with diacetyl monoxime is not limited to the antipyrine-containing reagent solution. Reagent A and reagent B are stored in storage tanks 41 and 42, respectively.

As already disclosed in Patent Literature 2, the present inventors have found that the peak intensity in absorbance measurement decreases when these reagents are prepared and then kept at room temperature for a long period of time (e.g., several days or more) for continuous quantification of urea, and that this decrease in peak intensity can be prevented by refrigerating the reagents (especially reagent B). In order to perform stable quantification, it is preferable that the peak intensity in the absorbance measurement does not decrease, so in the analyzing apparatus of the exemplary embodiment, storage tanks 41 and 42 are provided inside refrigeration section 40. Reagent A is prepared by dissolving diacetyl monoxime in an acetic acid solution, and if refrigeration section 40 is provided, the preparation itself is carried out in storage tank 41, or reagent A is stored in storage tank 41 after its preparation. Similarly, reagent B is prepared by dissolving antipyrine in, for example, sulfuric acid, and the preparation itself is carried out in storage tank 42, or reagent B is stored in storage tank 42 after its preparation. Refrigeration section 40 shields storage tanks 41, 42 from light and cools storage tanks 41, 42, thereby maintaining the temperature of reagents A and B in storage tanks 41, 42 at 20° C. or below, preferably at 3° C. or above and 20° C. or below, and more preferably at 5° C. or above and 15° C. or below. Storage tank 41 for storing reagent A does not necessarily have to be placed inside refrigeration section 40 as long as it can be stored in a light-shielded state. There is no problem even if the refrigeration temperatures of the reagents are less than 5° C. as long as crystals do not precipitate in the reagents. In the Methods of Analysis in Health Science (Non-Patent Literature 1), it is stated that an antipyrine sulfate solution prepared by dissolving antipyrine in sulfuric acid can be used for 2 to 3 months if stored in a brown bottle, and that refrigerated storage is not suitable because crystals will precipitate and will not redissolve even when returned to room temperature. However, the present inventors have confirmed through experiments that an antipyrine sulfate solution prepared according to the Methods of Analysis in Health Science does not crystallize even at 3° C. If a dilution operation is carried out when preparing reagent A and reagent B, it is preferable to use urea-free water produced in accordance with the present invention as the water used for dilution.

One end of line 26 is connected to storage tank 41, and the other end of line 26 is connected to line 24 via mixing section 43. Line 26 is provided with pump P₂ for feeding reagent A into line 24 at a predetermined flow rate. Similarly, one end of line 27 is connected to storage tank 42, and the other end of line 27 is connected to line 24 via mixing section 44. Line 27 is provided with pump P₃ for feeding reagent B into line 24 at a predetermined flow rate. Mixing sections 43 and 44 function to uniformly mix reagent A and reagent B, respectively, with the liquid flow in line 24. The other end of line 24 is connected to the inlet of reaction coil 31 provided in reaction thermostatic chamber 30. Reaction coil 31 serves to cause a color reaction between urea and diacetyl monoxime in the presence of antipyrine inside it, and its length and the flow rate within reaction coil 31 are appropriately selected according to the residence time required for the reaction. Reaction thermostatic chamber 30 heats reaction coil 31 to a temperature suitable for the reaction, for example, to a temperature of 50° C. or above and 150° C. or below, preferably 90° C. or above and 130° C. or below.

Detector 32 is provided at the end, i.e., outlet, of reaction coil 31 for measuring the absorbance of the color produced in the liquid by the color reaction, for the liquid flowing out of reaction coil 31. The peak intensity or peak area of absorbance at a wavelength of about 460 nm, for example, is determined by detector 32. By using the absorbance when the carrier water is flowing as a baseline, and obtaining a calibration curve from the absorbance for a standard solution with a known urea concentration, the urea concentration in the sample water can be determined from the absorbance for the sample water. At the outlet of detector 32, back pressure coil 33 is provided for applying back pressure to the line from pump P₁ through sampling valve 10, line 24 and reaction coil 31 to detector 32. Pressure gauge PI₀ is connected to a position between the outlet of detector 32 and the inlet of back pressure coil 33. The waste liquid of the analyzing apparatus flows out of the outlet of back pressure coil 33.

When quantifying urea using the analyzing apparatus of the exemplary embodiment, it is necessary to prepare a calibration curve using a urea standard solution in advance. When preparing a urea standard solution to be used for preparing a calibration curve, urea-free water having a urea concentration of less than 1 μg/L is used, which is obtained by treating with added urease and then separating the remaining urease using a separation membrane, preferably a reverse osmosis membrane.

According to the analyzing apparatus of the exemplary embodiment, the urea contained at μg/L level in the sample water can be more accurately quantified since flow injection analysis is performed using a carrier water containing almost no urea, that is, has a urea concentration of less than 1 μg/L, particularly less than 0.5 g/L.

### EXAMPLE

Hereinafter, the present invention will be described in more detail below with reference to examples, but the present invention is not limited to these.

The separation membranes used in the examples and comparative examples are as follows.

### Reverse Osmosis Membrane

TW30 (product name: FilmTec TW30-1812-50 HR, manufactured by DuPont)
   Salt rejection rate: 99% (250 ppm NaCl, 25° C., pH7, Recovery rate 15%, Inlet pressure 0.34 MPa)
BW60 (product name: FilmTec BW60-1812-75, manufactured by DuPont)
   Salt rejection rate: 97% (250 ppm NaCl, 25° C., pH7, Recovery rate 15%, Inlet pressure 0.34 MPa)

### Nanofiltration Membrane

XT2 (product name: XT2-1812TM, manufactured by Synder FilRation)
   Molecular weight cutoff: 1,000 Da
   Salt rejection rate: 1.5% (250 ppm NaCl, 25° C., pH7, Recovery rate 15%, Inlet pressure 0.34 MPa)

### Experimental Example 1

Five milliliter of 10 mg/L urea solution was added to 5 L of ultrapure water to prepare 10 μg/L urea solution. To 5 L of the prepared urea solution, 1 mL of a urease solution (manufactured by Fujifilm Wako Pure Chemicals Corporation) with an enzyme activity of 150 U/mg was added to prepare a urease-added solution with a urease concentration of 500 μg/L. The urease-added solution thus prepared was stirred and then allowed to stand at room temperature. Sampling was performed at each time point, and the urea concentration was measured using an on-line urea meter (ORUREA (registered trademark), manufactured by Organo Corporation). The results are shown in Table 1. After 3.5 hours, the concentration was less than 0.5 μg/L and no urea was detected.

TABLE 1

| Time (hr) | Urea Concentration (μg/L) |
|---|---|
| 0 | 10 |
| 1 | 2.7 |
| 2 | 0.8 |
| 3.5 | <0.5 |
| 4 | <0.5 |

### Experimental Example 2

Eighty liters of the treated water containing urease, in which urea was decomposed, prepared according to the procedure of Experimental Example 1 and standing for at least 3.5 hours was treated in the test system shown in FIG. 2. In this case, BW60, a reverse osmosis membrane, was used as the separation membrane. The inlet flow rate was 1200 mL/min, the concentrated flow rate was 840 mL/min, and the permeate flow rate was 360 mL/min. The obtained concentrated water was circulated to the treated water before passing through BW60, and the passing of the treated water was continued until the amount of the treated water reached 4 L, and the inlet and outlet pressures of the separation membrane were measured. The inlet pressure was measured using pressure gauge PI₁ in FIG. 2. The outlet pressure refers to the pressure at the outlet of the concentrated water, and was measured using pressure gauge PI₂ in FIG. 2. The results are shown in Table 2. The higher the concentration rate, the more likely the membrane is to become clogged, but with a concentration of 20 times, operation was possible below the vessel's withstand pressure.

TABLE 2

| | Before | After |
|---|---|---|
| | Concentration (MPa) | Concentration (MPa) |
| Inlet Pressure | 0.47 | 0.52 |
| Outlet Pressure | 0.45 | 0.5 |

### Experimental Example 3

In the test system shown in FIG. 3(a), an unused reverse osmosis membrane was cleaned by passing urea-free water through it, and the concentrated water and permeate water discharged as waste liquid were sampled. The urea concentration of each of the sampled solutions was measured using the on-line urea meter (ORUREA (registered trademark), manufactured by Organo Corporation). This allowed us to observe the effect of using unused reverse osmosis membranes without cleaning them on the measurement of urea concentration. The results are shown in Table 3. The inlet flow rate was 1200 mL/min (0.50 MPa), the concentrated water flow rate was 840 mL/min (0.49 MPa), and the permeate water flow rate was 360 mL/min.

TABLE 3

| | Permeate | Concentrated |
|---|---|---|
| Cleaning Time (min) | Water (ppb) | Water(ppb) |
| 0 | 6.3 | <0.5 |
| 30 | <0.5 | <0.5 |
| 60 | <0.5 | <0.5 |

Immediately after the start of the liquid passage (cleaning time: 0 min), a peak of 6.3 ppb was detected in the measurement of the permeate water by the online urea meter. This indicates that using unused reverse osmosis membranes without cleaning them will affect the measurement of urea concentration. In the permeate water after 30 minutes of cleaning, no peak was detected by the online urea meter. Furthermore, no peak was detected by the online urea meter even after 60 minutes. This indicates that the 30-minute cleaning operation can eliminate the effect of using unused reverse osmosis membranes without cleaning them on the measurement of urea concentration.

### Example 1

Treated water containing urease, in which urea was not detected, prepared according to the procedure of Experimental Example 1 and standing for at least 3.5 hours was passed through the test system in FIG. 2. In this case, TW30, a reverse osmosis membrane, was used as the separation membrane. First, three-way valve 16 was operated as shown in FIG. 3(a), and the treated water was passed through according to the procedure of Experimental Example 3 to clean it so that the urea concentration measurement was not affected by the unused reverse osmosis membrane, and the concentrated water and the permeate water were discarded. Then, as shown in FIG. 3(b), three-way valve 16 was switched, 80 L of the treated water was passed through reverse osmosis membrane 15, and the concentrated water was circulated through concentrated water line 13 to treated water 8 until the treated water was concentrated to 4 L, thereby providing 20 times concentration. The inlet flow rate to reverse osmosis membrane 15 was 400 mL/min (0.28 MP), the concentrated water flow rate was 280 mL/min (0.25 MP), and the permeate water flow rate was 120 mL/min.

Urea-free water 5 was collected and urea solution was added to make urea concentrations 50 ppb and 100 ppb. The urea solution with a urea concentration of 50 ppb is referred to as Sample 1, and the urea solution with a urea concentration of 100 ppb is referred to as Sample 4. The solutions to which urea solution was added were allowed to stand at room temperature for several days, and changes in urea concentration were measured.

### Example 2

The change in urea concentration was measured in the same manner as in Example 1, except that BW60, a reverse osmosis membrane, was used as the separation membrane instead of TW30 used in Example 1. The urea solution with a urea concentration of 50 ppb is referred to as Sample 2, and the urea solution with a urea concentration of 100 ppb is referred to as sample 5. The inlet flow rate was 1200 mL/min (0.50 MPa), the concentrated water flow rate was 840 mL/min (0.49 MPa), and the permeate water flow rate was 360 mL/min.

### Comparative Example 1

The change in urea concentration was measured in the same manner as in Example 1 except that XT2, a nanofiltration membrane, was used as the separation membrane instead of TW30 used in Example 1. The urea solution with a urea concentration of 50 ppb is referred to as Sample 3, and the urea solution with a urea concentration of 100 ppb is referred to as Sample 6. The inlet flow rate was 400 mL/min (0.28 MP), the concentrated flow rate was 280 mL/min (0.25 MP), and the permeation flow rate was 120 mL/min.

TABLE 4

| | | | Number of Days elapsed (d) | | | | | | | |
|---|---|---|---|---|---|---|---|---|---|---|
| | Separation Membrane | Salt Rejection Rate | 1 | 4 | 5 | 7 | 10 | 13 | 15 | 27 |
| Sample No. | used | (%) | Urea Concentration (ppb) | | | | | | | |
| 1 | TW30 | 99 | 50 | 51 | — | 50 | — | 50 | — | — |
| 2 | BW60 | 97 | 50 | — | 50 | — | — | — | 50 | 51 |
| 3 | XT2 | 1.5 | 49 | 47 | — | — | 45 | — | — | — |

TABLE 5

| | | | Number of Days elapsed (d) | | | | | | | |
|---|---|---|---|---|---|---|---|---|---|---|
| | Separation Membrane | Salt Rejection Rate | 1 | 4 | 5 | 7 | 10 | 13 | 15 | 27 |
| Sample No. | used | (%) | Urea Concentration (ppb) | | | | | | | |
| 4 | TW30 | 99 | 100 | 102 | — | 101 | — | 100 | — | — |
| 5 | BW60 | 97 | 99 | — | 101 | — | — | — | 100 | 101 |
| 6 | XT2 | 1.5 | 98 | 96 | — | — | 95 | — | — | — |

In Tables 4 and 5, “-” indicates that the measurement was not performed. Samples 1, 2, 4 and 5, in which TW30 or BW60, a reverse osmosis membrane, was used, showed no change in urea concentration. In contrast, the urea concentration gradually decreased in samples 3 and 6, in which XT2, a nanofiltration membrane, was used. This is because urease permeated through XT2 and reacted with urea in the solution after urea was added.

From the above, it was found that TW30 and BW60, reverse osmosis membranes, were able to prevent urease from leaking into urea-free water, but XT2, a nanofiltration membrane, was unable to sufficiently prevent urease leakage.

### Example 3

The urea concentration of each of the urea standard solutions adjusted to a urea concentration of 0 to 50 μg/L was measured using an on-line urea meter (ORUREA (registered trademark), manufactured by Organo Corporation). In the measurements, the urea-free water prepared in Example 1 was used as carrier water 1 and the urea-free water prepared in Example 2 as carrier water 2.

TABLE 6

| | Separation | Urea Standard Solution (μg/L) | | | | | | |
|---|---|---|---|---|---|---|---|---|
| | Membrane | 0.0 | 1.0 | 2.0 | 5.0 | 10.0 | 20.0 | 50.0 |
| | used | Urea Concentration (μg/L) | | | | | | |
| Carrier | TW30 | 0.0 | 1.1 | 2.0 | 4.9 | 10.3 | 20.5 | 50.0 |
| water 1 | | | | | | | | |
| Carrier | BW60 | 0.0 | 1.1 | 2.0 | 5.0 | 10.1 | 20.2 | 50.0 |
| water 2 | | | | | | | | |

The error in the urea concentrations measured by the on-line urea meter using carrier waters 1 and 2 relative to the urea standard solutions adjusted to a urea concentration of 0 to 50 μg/L was within +5%. This indicates that the urea-free water prepared by the method according to the present invention can be applied to the analysis of low-concentration urea as a carrier water.

While the present invention has been described with reference to the exemplary embodiments, the present invention is not limited to the above exemplary embodiments. Various changes that can be understood by those skilled in the art may be made to the constitution and details of the present invention, within the scope of the present invention.

This application claims the benefit of priority from Japanese Patent Application No. 2022-38018, filed on Mar. 11, 2022, the disclosure of which is incorporated herein in its entirety by reference.

The present invention includes the following methods.

### [Method 1]

A method for producing urea-free water, comprising:
adding urease to a urea-containing water, thereby decomposing urea, to obtain a treated water containing urease, and
passing the treated water through a separation membrane, thereby removing the urease, to obtain a urea-free water.

### [Method 2]

The method for producing urea-free water according to Method 1, wherein the separation membrane is a reverse osmosis membrane.

### [Method 3]

The method for producing urea-free water according to Method 2, wherein, after obtaining a concentrated water containing urease using the reverse osmosis membrane, the treated water is concentrated by returning the concentrated water to the treated water.

### [Method 4]

The method for producing urea-free water according to Method 2 or 3, wherein the reverse osmosis membrane has a salt rejection rate of 90% or more.

### [Method 5]

The method for producing urea-free water according to any one of Methods 2 to 4, wherein the reverse osmosis membrane is used after being cleaned with a solution not containing urea.

### [Method 6]

A method for quantifying urea in sample water, comprising using a urea-free water produced by the method for producing urea-free water according to any one of Methods 1 to 5, as at least one of a water to be used in preparation of a urea standard solution, and a carrier water.

Also, the present invention includes the following configurations.

### [Configuration 1]

An apparatus for producing urea-free water, comprising:
a treatment means for decomposing urea by adding urease to a urea-containing water,
a separation membrane for removing the urease by passing a treated water obtained by the treatment means through the separation membrane,
wherein the water passing through the separation membrane is made into a urea-free water.

### [Configuration 2]

The apparatus for producing urea-free water according to Configuration 1, wherein the separation membrane is a reverse osmosis membrane.

### [Configuration 3]

The apparatus for producing urea-free water according to Configuration 2, further comprising a tank for storing the treated water, and a circulation path for returning a concentrated water from the reverse osmosis membrane to the tank.

### [Configuration 4]

An apparatus for analyzing urea, by introducing a constant amount of sample water into a flow of carrier water to quantify urea in the sample water,
the apparatus for analyzing urea comprising the apparatus for producing urea-free water according to any one of Configurations 1 to 3,
wherein a urea-free water having a urea concentration of less than 1 μg/L obtained from the apparatus for producing urea-free water is used as the carrier water.

### REFERENCE SIGNS LIST

101 Treatment means
102 Separation means
200 Apparatus for producing urea-free water
300 Apparatus for analyzing urea
1 Treated water
2 Urea-containing water
3 Urease
4 Separation membrane
5 Urea-free water not containing urease
8 Treated water containing urease in which urea is decomposed
9 Return line
13 Concentrated water line
14 Permeate water line
15 Reverse osmosis membrane.

1. A method for producing urea-free water, comprising:
adding urease to a urea-containing water, thereby decomposing urea, to obtain a treated water containing urease, and
passing the treated water through a separation membrane, thereby removing the urease, to obtain a urea-free water.

2. The method for producing urea-free water according to claim 1, wherein the separation membrane is a reverse osmosis membrane.

3. The method for producing urea-free water according to claim 2, wherein, after obtaining a concentrated water containing urease using the reverse osmosis membrane, the treated water is concentrated by returning the concentrated water to the treated water.

4. The method for producing urea-free water according to claim 2, wherein the reverse osmosis membrane has a salt rejection rate of 90% or more.

5. The method for producing urea-free water according to claim 2, wherein the reverse osmosis membrane is used after being cleaned with a solution not containing urea.

6. An apparatus for producing urea-free water, comprising:
a treatment means for decomposing urea by adding urease to a urea-containing water,
a separation membrane for removing the urease by passing a treated water obtained by the treatment means through the separation membrane,
wherein the water passing through the separation membrane is made into a urea-free water.

7. The apparatus for producing urea-free water according to claim 6, wherein the separation membrane is a reverse osmosis membrane.

8. The apparatus for producing urea-free water according to claim 7, further comprising a tank for storing the treated water, and a circulation path for returning a concentrated water from the reverse osmosis membrane to the tank.

9. A method for quantifying urea in sample water, comprising using a urea-free water produced by the method for producing urea-free water according to claim 1, as at least one of a water to be used in preparation of a urea standard solution, and a carrier water.

10. An apparatus for analyzing urea, by introducing a constant amount of sample water into a flow of carrier water to quantify urea in the sample water,
the apparatus for analyzing urea comprising the apparatus for producing urea-free water according to claim 6,
wherein a urea-free water having a urea concentration of less than 1 μg/L obtained from the apparatus for producing urea-free water is used as the carrier water.

11. A method for quantifying urea in sample water, comprising using a urea-free water produced by the method for producing urea-free water according to claim 2, as at least one of a water to be used in preparation of a urea standard solution, and a carrier water.

12. A method for quantifying urea in sample water, comprising using a urea-free water produced by the method for producing urea-free water according to claim 3, as at least one of a water to be used in preparation of a urea standard solution, and a carrier water.

13. A method for quantifying urea in sample water, comprising using a urea-free water produced by the method for producing urea-free water according to claim 4, as at least one of a water to be used in preparation of a urea standard solution, and a carrier water.

14. A method for quantifying urea in sample water, comprising using a urea-free water produced by the method for producing urea-free water according to claim 5, as at least one of a water to be used in preparation of a urea standard solution, and a carrier water.

15. An apparatus for analyzing urea, by introducing a constant amount of sample water into a flow of carrier water to quantify urea in the sample water,
the apparatus for analyzing urea comprising the apparatus for producing urea-free water according to claim 7,
wherein a urea-free water having a urea concentration of less than 1 μg/L obtained from the apparatus for producing urea-free water is used as the carrier water.

16. An apparatus for analyzing urea, by introducing a constant amount of sample water into a flow of carrier water to quantify urea in the sample water,
the apparatus for analyzing urea comprising the apparatus for producing urea-free water according to claim 8,
wherein a urea-free water having a urea concentration of less than 1 μg/L obtained from the apparatus for producing urea-free water is used as the carrier water.
